(19) United States
(12) Patent Application Publication
(10) Pub. No.: US 2024/0207388 A1
(43) Pub. Date: Jun. 27, 2024

(54) Methods for Providing a Vaccine

(71) Applicants: Wolfgang WÜRFEL, Sixtnitgern/Odelzhausen (DE); Franziska WÜRFEL, Sixtnitgern/Odelzhausen (DE)

(72) Inventors: Wolfgang WÜRFEL, Sixtnitgern/Odelzhausen (DE); Franziska WÜRFEL, Sixtnitgern/Odelzhausen (DE)

(21) Appl. No.: 17/913,822

(22) PCT Filed: Mar. 25, 2021
(86) PCT No.: PCT/EP2021/057746

§ 371 Date: Sep. 22, 2022

(30) Foreign Application Priority Data
Mar. 26, 2020 (DE) 10 2020 108 453.6

(51) Int. Cl. A61K 39/215; A61K 39/00; C12N 7/00

(52) U.S. Cl. CPC A61K 39/215; C12N 7/00; A61K 2039/5252; A61K 2039/544; A61K 2039/55505; C12N 2770/20034; C12N 2770/20051; C12N 2770/20061

(57) ABSTRACT

Methods for producing and providing a vaccine for immunizing an individual against an illness caused by a virus are provided, including viruses from the family of coronaviruses. The method includes obtaining a sample of the virus; inactivating the virus by destroying or removing nucleic acids carrying genetic information for the virus; and preparing the inactivated virus in order to obtain an administrable vaccine to be administered. Vaccines provided in such a manner and the use thereof are also provided.

### FIELD OF THE INVENTION

The present invention relates to the field of providing vaccines for immunizing an individual against an illness caused by a virus from the family of coronaviruses.

### BACKGROUND OF THE INVENTION

According to the current state of knowledge, the novel SARS-COV-2 coronavirus, which can lead to the illness Covid-19, is transmitted by droplet infection and can lead to an infection of the upper and lower respiratory tract. Such an infection leads to immunity relatively quickly.

The respiratory tract is very well protected immunologically by what is referred to as the Waldeyer's ring. This includes various lymphoid organs, such as the pharyngeal tonsils (“tonsils”), palatine tonsils, and lingual tonsil, which are grouped together in a quasi-circle around the pharynx, down to the voice box (larynx). Such a “structure” of lymphoid organs can also be found at other body entry points, since these represent natural “gateways” for infections.

A distinction should be made between the colonization of body tissues, e.g., with a virus, and the outbreak of an infection, i.e., the inflammatory reaction of the immune system with clinical symptoms.

Viruses are not capable of reproducing on their own. They need a host that will carry out this reproduction for them. The novel coronavirus also docks onto host cells, in this case human cells in the respiratory tract, smuggles in its RNA, and ultimately induces, via the cell infected in this way, an intracellular synthesis process which aims to have all the “building blocks” of the virus reduplication produced by “outsourcing.” In the end, various virus copies are provided and released through degeneration of the affected cell.

In this way, the organism is again invaded by numerous viruses, which contributes to accentuating the clinical symptoms. The clinical symptoms are largely determined by the reaction of the immune system. Intracellular peptides or antigens are presented on the cell surface, in particular by the class II HLA groups. The intracellular virus attack changes the intracellular protein signature and thus also the peptides or antigens presented by the HLA groups. If an immune cell (e.g., T-lymphocyte) identifies a virus-infected cell as “not normal”, this cell is attacked, via inflammatory cascade, with the aim of eliminating this cell.

This also applies to infections with the novel coronavirus. The problem here is that the specific reaction of the immune system can lead to a kind of granulomatous pneumonia (similar to tuberculosis), apparently particularly when there has been no immunization by means of the Waldeyer's ring (e.g., in immunosuppressed or less immunocompetent individuals from groups at risk). Such pneumonia can lead very quickly to respiratory failure, which is largely responsible for the reported deaths.

But regardless of the clinical course of the disease: immunity (IgG, IgA, and IgM antibodies against Covid-19) can only develop against surface structures/characteristics (proteins) of the virus, and certainly not against its specific RNA.

Vaccines against novel viruses are generally not available when the virus appears. Conventional vaccinations have some disadvantages: they cannot be implemented easily and rapidly with new types of viruses. Insofar as they act intrinsically (e.g., through injection), they have to overcome high and therefore lengthy hurdles for approval. Mass production is often complex.

Against this background, the object of the invention is that of providing methods for providing a vaccine for immunizing an individual against an illness caused by a virus, in particular against an illness transmitted via the respiratory tract, vaccines of this type, and the use of such vaccines, while avoiding intrinsic manipulation.

### DESCRIPTION OF THE INVENTION

This object is achieved using methods, vaccines, and uses according to the independent claims. The dependent claims describe preferred embodiments.

According to a first aspect, methods for providing a vaccine are provided. The vaccine is used to immunize an individual against an illness caused by a virus from the family of coronaviruses. In one step of the method, a sample of the virus is obtained. The method further comprises inactivating the virus such that nucleic acids carrying genetic information for the virus are destroyed or removed. In addition, the inactivated virus is prepared in order to obtain an administrable vaccine to be administered as an inhalable aerosol or as a solution that can be gargled.

In particular, the virus can be SARS-COV-2.

In some embodiments the method also comprises culturing the virus of the sample obtained, in particular in a cell culture.

The virus can be inactivated, for example by irradiating the sample, in particular with UV rays.

In some embodiments, inactivating the virus may comprise applying a hypoosmolar environment.

In particular, after applying the hypoosmolar environment, the nucleic acids of the virus can be removed.

In some embodiments, the method may further comprise enriching the vaccine with an immunization enhancer, e.g., aluminum hydroxide.

In a second aspect, the invention relates to a vaccine provided using a method according to the first aspect.

In a third aspect, the invention relates to the use of a vaccine according to the second aspect. For example, the use may include administration for single or repeated inhalation of the aerosol.

### BRIEF DESCRIPTION OF THE DRAWING

FIG. 1 shows a flow chart of a method according to an exemplary embodiment.

### DESCRIPTION OF EXEMPLARY EMBODIMENTS

Methods for providing a vaccine for immunizing an individual against an illness caused by a virus from the family of coronaviruses are described.

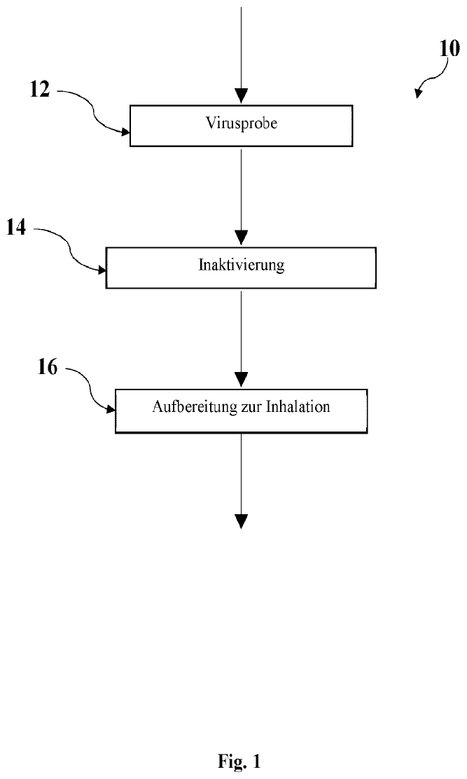

FIG. 1 shows a method 10 for providing a vaccine for immunizing an individual against an illness caused by a virus from the family of coronaviruses.

The method, described by way of example, induces immunity against the illness Covid-19 without leading to a clinically apparent or severe infection (above all without pneumonia). In addition to this specific example, the methods described can be used for all viral illnesses from the family of coronaviruses that are transmitted by means of droplet infection and the respiratory tract.

Individuals who become infected with the novel coronavirus and develop, e.g., laryngitis, are usually very well protected against pneumonia (which can be critical in other cases). Organs of the Waldeyer's ring can substantially immunologically intercept the invasion of the virus. In such cases, inflammation develops, as a result of which immunity builds up relatively quickly.

Structural features of SARS-COV-2 are: the genetic material is a single-strand DNA or RNA with 29,903 nucleotides. It encodes 10 proteins, with the S1 protein being responsible for binding the virus to a cell and the S2 protein being responsible for fusing with the cell membrane. These protein structures protrude like “spikes” from the fatty virus envelope, hence the designation “S1” or “S2.” These spikes are approximately 9-12 nm long and binding to the host cell's ACE-2 receptor is essential for the virus to mediate infection/reduplication.

### Immunization Process:

In one step 12, a sample of the virus is obtained. The virus is reproduced to create a suitable cell culture.

In one step 14, the virus is inactivated so that nucleic acids carrying genetic information for the virus are destroyed or removed. The virus, and in particular its RNA, are inactivated using suitable measures (e.g., using irradiation). Alternatively, this can be achieved, e.g., by allowing the virus to swell in hypoosmolar solution, causing it to burst. The RNA is removed and the fragments of the envelope are preserved.

In one step 16, the inactivated virus or the virus envelope fragments are prepared in an aerosol for inhalation.

The aerosol is enriched with an immunization enhancer, e.g., aluminum hydroxide.

Immunization takes place by inhaling the aerosol, which can be repeated several times depending on the immunization effect. The aerosol can be inhaled by means of a spray, for example using an inhaler or a nasal spray.

The manner of application can be modified: e.g., a gargle solution is possible so that the lower respiratory tract remains unaffected. Conversely, if it makes sense and is necessary, “ultranebulizers” can be used to produce very small droplets that can then penetrate deeper into the respiratory tract.

This procedure can be adapted according to the individual reaction of the immune system (anti-Covid-19 antibody testing of immunoglobulins of M, A, and G classes in the blood), so unlimited repeated immunization cycles are possible. No clinical symptoms or very few clinical symptoms are to be expected, and if there are any, they are expected to be only very minor. Nevertheless, individual immunization can be achieved.

The complete, but RNA-inactivated, virus will not trigger an immune response as intense as the native, infectious virus. This is also not necessary, since discreet immunization is also helpful (what is referred to as “occult immunization”). In any case, the effect can be intensified in a very dosed manner by increasing the number of sprays.

Envelope fragments will elicit a stronger immune response than an intact envelope. This is because fragments present themselves to the immune system from “front and back,” i.e., from outside and inside. This increases the antigenic stimulus. The antigenic stimulus can also be increased by binding the fragments to proteins with a strong antigenic effect, e.g., those of animal origin (e.g., bovine serum albumin).

The concept presented aims for immunization via body surfaces (“extrinsic”), albeit internal body surfaces (respiratory tract, Waldeyer's ring). Previous immunization concepts bring the antigen, against which immunization is intended to be achieved, into the body (“intrinsically”). Intrinsic immune manipulations have a higher risk potential, e.g., for (undesirable) side effects, than extrinsic ones.

What is claimed is:

1. A method for providing a vaccine for immunizing an individual against an illness caused by a virus from the family of coronaviruses, comprising:
obtaining a sample of the virus;
inactivating the virus so that nucleic acids carrying genetic information for the virus are destroyed or removed; and,
preparing the inactivated virus in order to obtain an administrable vaccine to be administered as an inhalable aerosol or as a solution that can be gargled.

2. The method according to claim 1, wherein the virus is SARS-CoV-2.

3. The method according to claim 1, further comprising culturing the virus of the sample obtained.

4. The method according to claim 1, wherein inactivating the virus comprises irradiating the sample with UV rays.

5. The method according to claim 1, wherein inactivating the virus comprises applying a hypoosmolar environment.

6. The method according to claim 5, wherein, after the hypoosmolar environment is applied, the nucleic acids of the virus are removed.

7. The method according to claim 1, further comprising: enriching the vaccine with an immunization enhancer.

8. The method according to claim 7, wherein the immunization enhancer comprises aluminum hydroxide.

9. A vaccine for immunizing an individual against an illness caused by a virus from the family of coronaviruses created by a process comprising:
obtaining a sample of the virus;
inactivating the virus so that nucleic acids carrying genetic information for the virus are destroyed or removed; and,
preparing the inactivated virus in order to obtain an administrable vaccine to be administered as an inhalable aerosol or as a solution that can be gargled.

10. The method according to claim 1 further comprising administering the vaccine to the individual.

11. The method according to claim 10, wherein administering the vaccine includes for single or repeated inhalation of an aerosol.
